(19) United States
(12) Patent Application Publication
(10) Pub. No.: US 2012/0283804 A1
(43) Pub. Date: Nov. 8, 2012

(54) MID-INFRARED LASER THERAPY DEVICE AND SYSTEM

(75) Inventors: Jin U. Kang, Ellicott City, MD (US); Yong Huang, Baltimore, MD (US); Kang Zhang, Baltimore, MD (US)

(73) Assignee: The Johns Hopkins University, Baltimore, MD (US)

(21) Appl. No.: 13/464,784

(22) Filed: May 4, 2012

Related U.S. Application Data
(60) Provisional application No. 61/482,304, filed on May 4, 2011.

(51) Int. Cl. A61N 5/067; A61N 5/01

(52) U.S. Cl. 607/89

(57) ABSTRACT

A mid-infrared laser therapy tool includes a handle, a mid-infrared laser contained within the handle, and a light pipe optically coupled to the mid-infrared laser. The mid-infrared laser emits at a wavelength within a wavelength band from about 6.0 μm to about 6.5 μm. A mid-infrared laser therapy system includes a mid-infrared laser therapy tool, an optical coherence tomography system adapted to communicate with the mid-infrared laser therapy tool, and a motorized-platform control system adapted to communicate with the mid-infrared laser therapy tool and the optical coherence tomography system. The mid-infrared laser therapy tool comprises a mid-infrared laser emits at a wavelength within a wavelength band from about 6.0 μm to about 6.5 μm.

### CROSS-REFERENCE OF RELATED APPLICATION

This application claims priority to U.S. Provisional Application No. 61/482,304 filed May 4, 2011, the entire contents of which are hereby incorporated by reference.

This invention was made with Government support of Grant No. 102811, awarded by the National Science Foundation. The U.S. Government has certain rights in this invention.

### BACKGROUND

1. Field of Invention

The field of the currently claimed embodiments of this invention relates to laser therapy devices and systems, and more particularly to mid-infrared laser therapy devices and systems.

2. Discussion of Related Art

Surgical treatment of diabetic retinopathy and other retinal pathologies frequently requires peeling of thin membranes from the retinal surface. Proliferative vitreoretinopathy, the most common cause of failure after retinal reattachment surgery (˜10%), requires complete mechanical removal of the epiretinal scar tissue to relieve traction so that the retina can reattach. For macular puckers, removal of the epiretinal membrane overlying the central macula improves vision by relieving tractional folds (“wrinkles”) in the retina. A study showed that 40% of patients with epiretinal membrane improved to 20/40 or better following surgery with at least a 2 line visual improvement in 85% of the patients. The concept of membrane peeling was developed in 1972 soon after the invention of vitrectomy. Originally, peeling was done with the bent tip of a needle. Innovations in instrumentation now allow the surgeon to use fine blades to make small incisions when an “edge” of scar tissue is not identified, micro-forceps to grasp tissue, and mechanical scissors to cut adhesions with the retina. The goal of the surgery is to minimize mechanical trauma to the retina while completely relieving retinal traction by removing epiretinal membranes.

The development of vitrectomy surgery for the treatment of macular hole is one of the most important events in the history of vitreous surgery. From its first description in 1869, macular hole was considered an untreatable cause of blindness. Since then, hundreds of thousands of patients worldwide have benefited from this remarkable surgery. One of the key innovations leading to improved success rates has been internal limiting membrane (ILM) peeling. There is no edge to the ILM to lift as in epiretinal membrane peeling. Therefore, a required element is to incise the ILM without injuring the underlying retina. The thickness of the ILM is 1-3 microns and is indistinguishable from deeper layers of the retina, making the optimum depth incision very difficult.

These maneuvers require excellent visualization (lighting, magnification, resolution, depth perception etc.) and precise instrument action (diamond dusted end gripping forceps or other precisely aligned tip forceps). In addition, current retina laser therapy procedures use visible wavelengths or near-IR lasers that are not precisely targeted and cause a large amount of collateral thermal damages to the surrounding retina tissues/layers. Therefore, there remains a need for improved laser therapy devices and systems.

### SUMMARY

A mid-infrared laser therapy tool according to an embodiment of the current invention includes a handle, a mid-infrared laser contained within the handle, and a light pipe optically coupled to the mid-infrared laser. The mid-infrared laser emits at a wavelength within a wavelength band from about 6.0 μm to about 6.5 μm.

A mid-infrared laser therapy system according to an embodiment of the current invention includes a mid-infrared laser therapy tool, an optical coherence tomography system adapted to communicate with the mid-infrared laser therapy tool, and a motorized-platform control system adapted to communicate with the mid-infrared laser therapy tool and the optical coherence tomography system. The mid-infrared laser therapy tool comprises a mid-infrared laser emits at a wavelength within a wavelength band from about 6.0 μm to about 6.5 μm.

### BRIEF DESCRIPTION OF THE DRAWINGS

Further objectives and advantages will become apparent from a consideration of the description, drawings, and examples.

FIG. 1 is a schematic illustration of a mid-infrared laser therapy tool according to an embodiment of the current invention.

FIG. 2 is a schematic illustration of a mid-infrared laser therapy system according to an embodiment of the current invention.

FIG. 3 is a schematic illustration showing an example of the use of a mid-infrared laser therapy tool and system according to an embodiment of the current invention.

FIGS. 4A-4C show examples of (a): Camera Image of Cow Eye Retinal Photocoagulation 5 positions were coagulated. (b): OCT Image of position 1 after long exposure (c): OCT Image of position 3 two tiny surface coagulation points after short exposure.

FIGS. 5A-5C show examples of (a): OCT en-face image of five laser burning points on sheep blood surface, (b): OCT en-face image of one laser burning point (circle marked), (c): OCT side view projection of burning point in (b).

FIG. 6 provides experimental data to help explain some concepts of the current invention, as follows: (a) experiment setup; (b1)-(b4)×250 digital optical microscope images correspond to (1)-(4) laser operation condition; (b5)-(b8)×750 digital optical microscope images of (b1)-(b4) with feature sizes marked; (c1)-(c2) two orthogonal cross-sectional OCT images of the ablation line at different positions; (c3) axial cross-sectional OCT image of the ablation line (White dashed squares were marked on the image to indicate areas where the craters were); (c4) is the enface projection of the volume, ablation lines was marked by arrows on the image.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Some embodiments of the current invention can provide a method of intraocular laser therapy using a quantum cascaded laser operating around 6 micron wavelength regime. According to an embodiment of the current invention, a light pipe is used to deliver a mid-IR beam directly to intraocular tissues that require a laser procedure. The method is suitable for procedures such as tissue ablation, coagulation, and welding. In an example, we used a 6.2 micron QCL laser and demonstrated efficient retina ablation and coagulation with minimal collateral damage to the surrounding tissue. Some embodiments of the current invention can also include an optical coherence tomography sensor to detect the tissue surface and control the position of the waveguide tip for highly precise laser power delivery control.

Some embodiments of the current invention can provide retinal surgeons with a new highly precise and effective surgical tool based on mid-IR laser abrasion to remove epiretinal and ILM and perform other types of laser therapy such as coagulation that can enhance their ability to achieve surgical objectives, diminish surgical risk and improve outcomes.

An embodiment of the current invention uses ˜6.3 μm QCL for epiretinal and internal limiting membrane abrasion, and other laser therapeutic procedures. It uses a ˜10 cm long QC light delivery pipe which includes an end-fire coupling of a QC laser to the light pipe and focusing optics at the distal end of the light pipe for laser therapy. A short waveguide section can be provided following the QCL lasing facet, which can provide a compact and high coupling efficiency to the light pipe while protecting the lasing facet. A combined optical coherence tomography (OCT) and motor control system can control the distance of the probe tip and the retina surface with an accuracy of around 1 micron.

Mid-IR wavelengths around 6 micron cause a minimal amount of collateral damage due to the OH absorption induced deformation and strong protein absorption which maintains high local heat concentration that is independent of surrounding tissue temperature and water content. This can allow precise ablation, coagulation and welding of retina with minimal collateral damage.

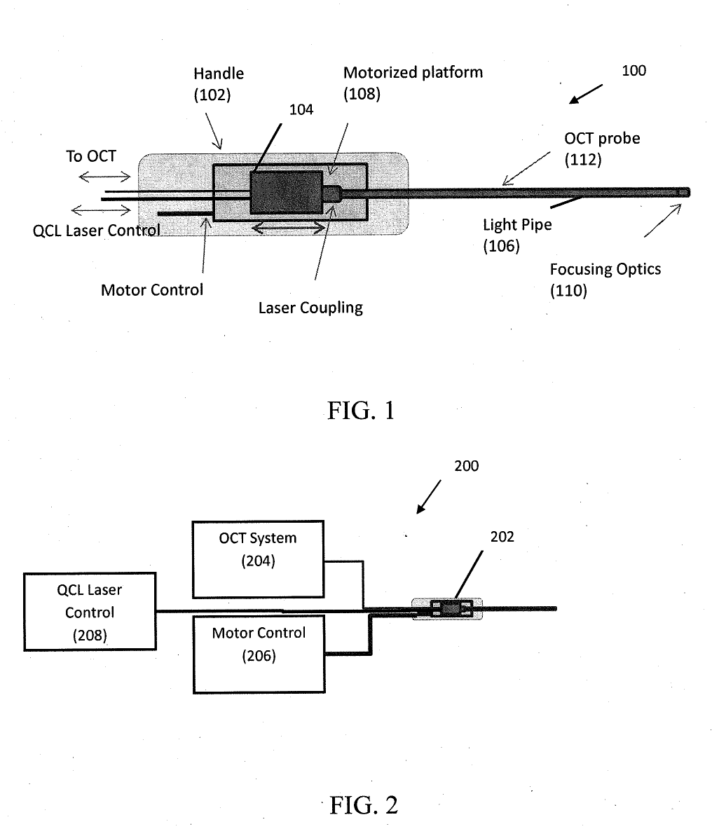

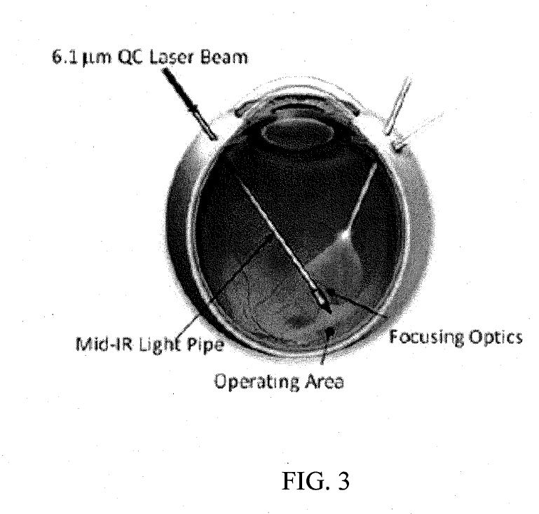

FIG. 1 is a schematic illustration of a mid-infrared laser therapy tool 100 according to an embodiment of the current invention. The mid-infrared laser therapy tool 100 has a handle, a mid-infrared laser 104 contained within the handle 102, and a light pipe 106 optically coupled to the mid-infrared laser 104. The mid-infrared laser 104 emits at a wavelength within a wavelength band from about 6.0 μm to about 6.5 μm. The term “about” here means to with ±0.1 μm. In some embodiments, the mid-infrared laser emits at a wavelength either at 6.1 μm or 6.45 p.m. At 6.1 μm, absorption by collagen is about double that of water. At 6.45 μm absorption by collagen is about six times greater than that of water. The mid-IR absorption of both water and collagen fall off gradually with increasing wavelength until around 10 μm, although collagen does have local absorption maxima at about 7 μm and 8 μm. However, the absorption at these local peaks is still much lower than that at either 6.1 or 6.45 μm. For wavelengths shorter than 6.1 μm, the absorption is mostly due to water and falls off rapidly. Consequently, the above-noted range is suitable for application in which tissue damage due to rapid heating of water in the tissue should be avoided. A quantum cascade laser is suitable for use according to some embodiments of the current invention. However, lasers that emit in other wavelength ranges may be used according to other embodiments of the current invention.

The mid-infrared laser therapy tool 100 can be hand-held by a surgeon or therapist. However, in other embodiments, it can be attachable to a robotic system for automated and/or semi-automated applications.

The light pipe 106 can be a hollow core waveguide, for example. For the mid-IR range of about 6.0 μm to about 6.5 μm there currently are no suitable optical fibers due to the strong absorption of currently available materials at those wavelengths.

In some embodiments of the current invention, the mid-infrared laser therapy tool 100 can also include a motorized platform 108 attached within the handle 102. The motorized platform 108 includes a movable component such that the mid-infrared laser 104 is attached to the movable component of the motorized platform 108. The motorized platform 108 can move the mid-infrared laser 104 and the light pipe 106 at least back and forth in a longitudinal direction to alter a distance of a distal end of the light pipe 106 from a surface of tissue to be irradiated. The motorized platform can be a platform such as that described in international PCT application no. PCT/US2011/044693, published as WO 2012/012540 A2 which is assigned to the same assignee as the current application, the entire content of which is hereby incorporated by reference for all purposes.

The mid-infrared laser therapy tool 100 can also include a focusing optical unit 110 arranged at a distal end of the light pipe 106 to focus mid-infrared light emitted from the light pipe 106.

In further embodiments of the current invention, the mid-infrared laser therapy tool 100 can also include an optical fiber 112 attached to the light pipe 106 having a distal end proximate and fixed relative to a distal end of the light pipe 106. The optical fiber 112 can be a probe for an OCT system, for example, to determine a distance to a surface of tissue to be irradiated. The motorized platform 108 can be adapted to communicate with a motor control system that is adapted to communicate with the optical coherence tomography system such that the motorized platform 108 can be moved to maintain a substantially constant distance from a surface of tissue being irradiated.

FIG. 2 is a schematic illustration of a mid-infrared laser therapy system 200 according to an embodiment of the current invention. The mid-infrared laser therapy system 200 includes a mid-infrared laser therapy tool 202, an optical coherence tomography system 204 adapted to communicate with the mid-infrared laser therapy tool 202, and a motorized-platform control system 206 adapted to communicate with the mid-infrared laser therapy tool 202 and the optical coherence tomography system 204. The mid-infrared laser therapy tool 202 includes a mid-infrared laser that emits at a wavelength within a wavelength band from about 6.0 μm to about 6.5 μm. The mid-infrared laser therapy tool 202 can be the same or similar to the embodiments described above. In addition, the OCT system 204 and motorized-platform control system 206 can use systems described in PCT/US2011/044693 incorporated by reference above. In addition, the mid-infrared laser therapy system 200 can also include a laser control system 206.

### Example 1

The following examples are provided to help explain some concepts of the current invention. The broad concepts of the current invention are not limited to these specific examples.

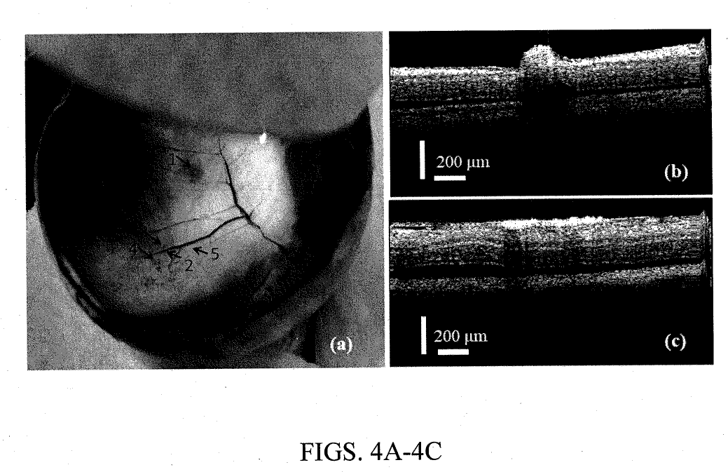

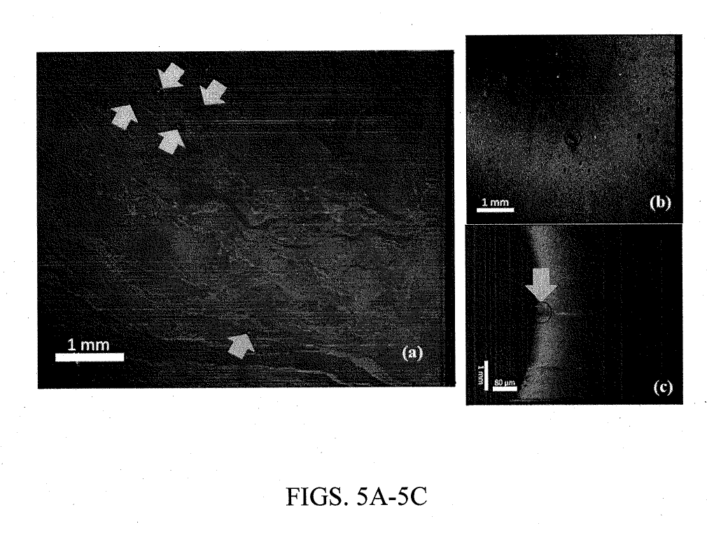

Some preliminary ex-vivo cow eye retina photocoagulation and sheep blood ablation results using 6.2 μm QCL are shown in FIGS. 4A-4C and FIGS. 5A-5C, respectively. The results show potential of ˜6 μm QCL for intraocular ablation and photocoagulation.

### Example 2

Lasers have shown an enormous potential for creating high precision surgical instruments since laser radiation can be focused into a small spot, selectively, and are strongly absorbed by tissue. Effectiveness of UV ArF excimer lasers has been proven for ablation of corneal stroma, which is used during laser refraction surgery to reshape the curvature of cornea. However, standard fiber light guides can't be used to deliver UV light and the mutagenic properties of the short wave UV radiation limits its use for ablation of other tissues (V. A. Serebryakov, É. V. Bo\check{i}ko, N. N. Petrishchev, and A. V. Yan, “Medical applications of mid-IR lasers. Problems and prospects,” J. Opt. Technol. 77, 6-17 (2010)). High absorption properties of bio-tissues in the IR range makes IR lasers a good choice for high-precision bio-tissue ablation. Thus suitable Mid-IR light sources that can meet the requirements of clinical and medical applications are provided according to some embodiments of the current invention. 6.1 μm Mid-IR lasers have been studied due to its advantage of high effectiveness and minimal collateral damages. Quantum cascade lasers have advantages of low cost, compact size and tunable wavelength (Scott S. Howard, Zhijun Liu, and Claire F. Gmachl, “Thermal and Stark-effect Roll-over of Quantum Cascade Lasers” IEEE Journal of Quantum Electronics 44 (4) 319-323 (2008)), which makes them a great Mid-IR light source alternative to contemporary tunable mid-IR sources such as free-electron lasers (FEL). In this example, we performed a bovine corneal ablation study in-vitro using a high-power pulsed 6.1 μm quantum cascade laser (QCL) and show that effective cornea stroma ablation was achieved.

### EXPERIMENT AND RESULTS

The system setup is shown in FIG. 5 (See, upper left (a)). A C-mount quantum cascade laser (T0991-4) from AdTech Optics was mounted on a laser mount that was connected to a TEC controller and a laser driver. The QCL diode is very compact with a dimension of 6×6×4 mm. When operated in pulse mode at T=15° C., the central wavelength of light is 6.12 μm and the peak output power was 791.7 mW. Two CaF₂ mid infrared focal lenses were combined to form a focusing lens with an effective focal length of 12.5 mm to deliver the focused beam onto a cornea sample. The bovine cornea sample was rested on a three dimensional translation stage. Four different laser operating parameter sets were tested: (1) I=1320 mA, Pulse width=20 ms, Frequency=25 Hz; (2): I=1320 mA, Pulse width=10 ms, Frequency=25 Hz; (3) I=1320 mA, Pulse width=5 ms, Frequency=100 Hz; (4) I=1320 mA, Pulse width=5 ms, Frequency=10 Hz. When the laser driver current was 1320 mA, the effective peak optical power delivered to the cornea sample was 250 mW. The sample was manually driven laterally when the laser was irradiated on the sample to form a line shape ablation.

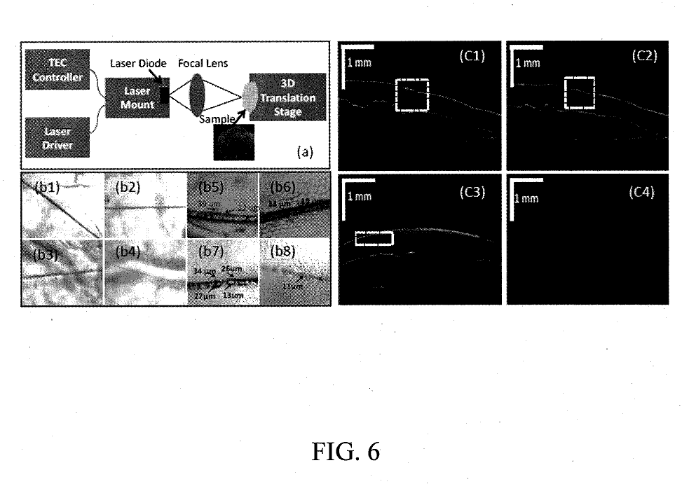

A digital optical microscope was used to observe the ablation lines of different conditions. FIGS. 6, (b1)-(b4) correspond to (1)-(4) laser operation sets, respectively. The magnification was ×250 for FIG. 6, (b1)-(b4). We can clearly see the ablation lines achieved on the corneal surface. The boundaries of the ablation lines were neat and clean, which showed minimal peripheral damage. To further quantify the collateral damage of the laser radiation, a zoomed view with 750 magnification of different ablation lines were shown in FIG. 6, (b5)-(b8), which corresponds to (b1)-(b4) respectively. The feature size of the ablation lines were marked on the images. As the pulse width decreased from 20 ms to 5 ms, the width of the ablation line decreased from 39 μm to 11 μm. The reason for this is that the energy of each pulse decreases when the pulse width is shortened and also the laser beam profile changes slightly when the pulse width changes. The depth of the ablation was estimated to be around 1-2 μm by the displacement that the sample moved when the top and bottom of the ablation line were at-focus separately. The depth corresponds to the optical penetration depth of 6.1 μm wavelength light, which is 2.79 μm (V. A. Serebryakov, É. V. Bo\check{i}ko, N. N. Petrishchev, and A. V. Yan, “Medical applications of mid-IR lasers. Problems and prospects,” J. Opt. Technol. 77, 6-17 (2010)). Fourier-domain optical coherence tomography (FD-OCT) with an axial resolution of 6 μm was also used to characterize the ablation lines. Due to extreme shallow depth of lines (b1)-(b3) which were beneath the resolution of OCT, no significant depth profile results were attained. However, as the pulse frequency increases from 10 Hz (b4) to 100 Hz (b3), craters with depth of 200 μm on the cornea stroma were observed by the OCT system. Images are shown in FIGS. 6, (c1)-(c4). From the OCT images, we can clearly see the craters that lie beneath the cornea surface.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A mid-infrared laser therapy tool, comprising:
a handle;
a mid-infrared laser contained within said handle; and
a light pipe optically coupled to said mid-infrared laser,
wherein said mid-infrared laser emits at a wavelength within a wavelength band from about 6.0 μm to about 6.5 μm.

2. A mid-infrared laser therapy tool according to claim 1, wherein said mid-infrared laser emits at a wavelength of one of 6.1 μm or 6.45 μm.

3. A mid-infrared laser therapy tool according to claim 1, wherein said mid-infrared laser is a quantum cascade laser.

4. A mid-infrared laser therapy tool according to claim 1, wherein said light pipe is a hollow core waveguide.

5. A mid-infrared laser therapy tool according to claim 1, further comprising a motorized platform attached within said handle, said motorized platform comprising a movable component,
wherein said mid-infrared laser is attached to said movable component of said motorized platform such that said motorized platform can move said mid-infrared laser and said light pipe at least back and forth in a longitudinal direction to alter a distance of a distal end of said light pipe from a surface of tissue to be irradiated.

6. A mid-infrared laser therapy tool according to claim 1, further comprising a focusing optical unit arranged at a distal end of said light pipe to focus mid-infrared light emitted from said light pipe.

7. A mid-infrared laser therapy tool according to claim 5, further comprising a focusing optical unit arranged at a distal end of said light pipe to focus mid-infrared light emitted from said light pipe.

8. A mid-infrared laser therapy tool according to claim 1, further comprising an optical fiber attached to said light pipe having a distal end proximate and fixed relative to a distal end of said light pipe,
wherein said optical fiber is adapted to be attached to an optical coherence tomography system to at least determine a distance to a surface of tissue to be irradiated.

9. A mid-infrared laser therapy tool according to claim 5, further comprising an optical fiber attached to said light pipe having a distal end proximate and fixed relative to a distal end of said light pipe,
wherein said optical fiber is adapted to be attached to an optical coherence tomography system to at least determine a distance to a surface of tissue to be irradiated, and
wherein said motorized platform is adapted to communicate with a platform control system, said platform control system being adapted to communicate with said optical coherence tomography system such that said motorized platform can be moved to maintain a substantially constant distance from a surface of tissue being irradiated.

10. A mid-infrared laser therapy tool according to claim 7, further comprising an optical fiber attached to said light pipe having a distal end proximate and fixed relative to a distal end of said light pipe,
wherein said optical fiber is adapted to be attached to an optical coherence tomography system to at least determine a distance to a surface of tissue to be irradiated, and
wherein said motorized platform is adapted to communicate with a motor control system, said platform control system being adapted to communicate with said optical coherence tomography system such that said motorized platform can be moved to maintain a substantially constant distance from a surface of tissue being irradiated.

11. A mid-infrared laser therapy system, comprising:
a mid-infrared laser therapy tool;
an optical coherence tomography system adapted to communicate with said mid-infrared laser therapy tool; and
a motorized-platform control system adapted to communicate with said mid-infrared laser therapy tool and said optical coherence tomography system,
wherein mid-infrared laser therapy tool comprises a mid-infrared laser emits at a wavelength within a wavelength band from about 6.0 μm to about 6.5 μm.

12. A mid-infrared laser therapy system according to claim 11, wherein said mid-infrared laser emits at a wavelength of one of 6.1 μm or 6.45 μm.

13. A mid-infrared laser therapy system according to claim 11, wherein said mid-infrared laser is a quantum cascade laser.

14. A mid-infrared laser therapy system according to claim 11, wherein said light pipe is a hollow core waveguide.

15. A mid-infrared laser therapy system according to claim 11, further comprising a motorized platform attached within said handle, said motorized platform comprising a movable component,
wherein said mid-infrared laser is attached to said movable component of said motorized platform such that said motorized platform can move said mid-infrared laser and said light pipe at least back and forth in a longitudinal direction to alter a distance of a distal end of said light pipe from a surface of tissue to be irradiated.

16. A mid-infrared laser therapy system according to claim 11, further comprising a focusing optical unit arranged at a distal end of said light pipe to focus mid-infrared light emitted from said light pipe.

17. A mid-infrared laser therapy system according to claim 15, further comprising a focusing optical unit arranged at a distal end of said light pipe to focus mid-infrared light emitted from said light pipe.

18. A mid-infrared laser therapy system according to claim 11, further comprising an optical fiber attached to said light pipe having a distal end proximate and fixed relative to a distal end of said light pipe,
wherein said optical fiber is adapted to be attached to an optical coherence tomography system to at least determine a distance to a surface of tissue to be irradiated.

19. A mid-infrared laser therapy system according to claim 15, further comprising an optical fiber attached to said light pipe having a distal end proximate and fixed relative to a distal end of said light pipe,
wherein said optical fiber is adapted to be attached to an optical coherence tomography system to at least determine a distance to a surface of tissue to be irradiated, and
wherein said motorized platform is adapted to communicate with a platform control system, said platform control system being adapted to communicate with said optical coherence tomography system such that said motorized platform can be moved to maintain a substantially constant distance from a surface of tissue being irradiated.

20. A mid-infrared laser therapy system according to claim 17, further comprising an optical fiber attached to said light pipe having a distal end proximate and fixed relative to a distal end of said light pipe,
wherein said optical fiber is adapted to be attached to an optical coherence tomography system to at least determine a distance to a surface of tissue to be irradiated, and
wherein said motorized platform is adapted to communicate with a platform control system, said platform control system being adapted to communicate with said optical coherence tomography system such that said motorized platform can be moved to maintain a substantially constant distance from a surface of tissue being irradiated.
